(19) United States
(12) Patent Application Publication
(10) Pub. No.: US 2022/0161283 A1
(43) Pub. Date: May 26, 2022

(54) DEVICE FOR CONTROLLING CONCENTRATION OF WATER-SOLUBLE SUBSTANCE IN SOLUTION

(71) Applicants: Steven S. Michalicek, Coralville, IA (US); Walter Neal Stanford, Social Circle, GA (US)

(72) Inventors: Steven S. Michalicek, Coralville, IA (US); Walter Neal Stanford, Social Circle, GA (US)

(21) Appl. No.: 17/530,920

(22) Filed: Nov. 19, 2021

Related U.S. Application Data
(60) Provisional application No. 63/118,225, filed on Nov. 25, 2020.

(51) Int. Cl. B05B 7/04; B05B 12/00; B05B 12/14

(52) U.S. Cl. CPC B05B 7/04; B05B 15/63; B05B 12/1418; B05B 12/002

(57) ABSTRACT

This disclosure recites a device that includes a body and a coupling near a first end of the body configured to attach to a water source. The device also includes interior tubing inside the body that includes a base portion that receives water into the device via the coupling, a fork that separates the base into a first path and a second path, and an end portion that combines the first and second paths. The device further includes a compartment along the second path that holds a solid, water-soluble substance. The device also includes a first dial that controls an amount of the water that enters the first and second paths. The device further includes a second dial that controls a rate that the water passes through the compartment. The device also includes a nozzle near a second end of the body.

### PRIORITY INFORMATION

This application claims the benefit of U.S. Provisional Application No. 63/118,225, filed Nov. 25, 2020, the entire contents of which are incorporated herein.

### TECHNICAL FIELD

The disclosure relates to sanitation devices.

### BACKGROUND

The COVID-19 pandemic has proven that daily habits must be adjusted to reduce the risk of exposure to various viruses, diseases, and other harmful elements. In order to maintain a healthy and effective society, focusing on cleaning and disinfecting public spaces, workplaces, businesses, schools, and homes is a valuable mitigation tactic. Reducing the risk of exposure to harmful elements by cleaning and disinfection is an important part of maintaining public spaces. The virus that causes COVID-19 can be killed if the right products are applied. The Environmental Protection Agency (EPA) has compiled a list of disinfectant products that can be used against COVID-19, including ready-to-use sprays, concentrates, and wipes. Each product has been shown to be effective against viruses that are harder to kill than viruses like the one that causes COVID-19.

### SUMMARY

In general, the disclosure is directed to a handheld device that is configured to attach to a water source, direct water through two separate portions of the device. One of those portions holds a solid, water-soluble substance. The device provide a connection to re-combine the water from the two separate portions to create a final solution before the final solution is sprayed out of an end nozzle. The device includes dials on each of the portions to control the amount of water and rate at which water passes over the water-soluble substance, thereby enabling the user to highly control the concentration of the water-soluble substance in the final solution before it is sprayed out into the user's immediate environment.

The device described herein provides a fast, easy, and safe device for many purposes, including sanitation of hard surfaces, where the user can dial-in the chlorine level that they desire for cleaning, sanitizing, or disinfecting. In addition to controlling chlorine levels, the device can be used with any water-soluble solid chemical to achieve exacting levels of solution for application. The device may be powered by normal water pressure (usually between 30 and 80 psi) with various nozzles for gentle sprays to jet streams up to 40 feet. The device may use solid, water-soluble substances, such as solid chlorine tablets, which provide erosion at predictable rates with highly accurate chlorine levels, eliminating premature dissolving and the safety issues associated with liquid chlorine.

Rather than mixing portions of an already created liquid solution into a single water stream, the device allows the user to “dial-in” and control chlorine levels that they desire. The device also may have built in chlorine test strips, so that the user can be assured of proper chlorine levels. The device may use chlorine tablets (e.g., 1″ chlorine tablets, or any other sized tablets) in a closed and metered chamber that assures consistency of chlorine delivery. The solid chlorine tablets deliver chlorine on demand in a closed chamber, thus eliminating fuming and the issues associated with liquid chlorine.

In one example, the disclosure is directed to a device comprising a body. The device further comprises a coupling configured to attach the device to a water source, the coupling near a first end of the body. The device also comprises interior tubing located inside the body, the interior tubing including a base portion, a fork that separates the base portion into a first path and a second path, and an end portion that combines the first path and the second path, wherein the base portion of the interior tubing receives water into the device via the coupling. The device further comprises a compartment along the second path that holds a solid, water-soluble substance. The device also comprises a first dial that, when rotated, controls an amount of the water that enters the first path from the base portion and an amount of the water that enters the second path. The device further comprises a second dial that, when rotated, controls a rate at which the water that enters the second path passes through the compartment such that the water combines with the solid, water-soluble substance held within the compartment to form a concentrated solution. The device also comprises a nozzle near a second end of the body, wherein the end portion of the interior tubing extends to the nozzle such that the water travelling through the first path of the interior tubing and the concentrated solution travelling through the second path of the interior tubing combines in the end portion of the interior tubing to form a final solution, and wherein the final solution exits the device through the nozzle.

In another example, the disclosure is directed to a system comprising a hose, a solid, water-soluble substance, and a device. The device comprises a body. The device further comprises a coupling configured to attach the device to the hose, the coupling near a first end of the body. The device also comprises interior tubing located inside the body, the interior tubing including a base portion, a fork that separates the base portion into a first path and a second path, and an end portion that combines the first path and the second path, wherein the base portion of the interior tubing receives water into the device via the coupling. The device further comprises a compartment along the second path that holds the solid, water-soluble substance. The device also comprises a first dial that, when rotated, controls an amount of the water that enters the first path from the base portion and an amount of the water that enters the second path. The device further comprises a second dial that, when rotated, controls a rate at which the water that enters the second path passes through the compartment such that the water combines with the solid, water-soluble substance held within the compartment to form a concentrated solution. The device also comprises a nozzle near a second end of the body, wherein the end portion of the interior tubing extends to the nozzle such that the water travelling through the first path of the interior tubing and the concentrated solution travelling through the second path of the interior tubing combines in the end portion of the interior tubing to form a final solution, and wherein the final solution exits the device through the nozzle.

In another example, the disclosure is directed to a method comprising rotating one or more of a first dial and a second dial of a device to adjust a concentration of one or more of a concentrated solution and a final solution. The device comprises a body. The device further comprises a coupling configured to attach the device to a water source, the coupling near a first end of the body. The device also comprises interior tubing located inside the body, the interior tubing including a base portion, a fork that separates the base portion into a first path and a second path, and an end portion that combines the first path and the second path, wherein the base portion of the interior tubing receives water into the device via the coupling. The device further comprises a compartment along the second path that holds a solid, water-soluble substance. The device also comprises the first dial, wherein rotating the first dial controls an amount of the water that enters the first path from the base portion and an amount of the water that enters the second path. The device further comprises the second dial, wherein rotating the second dial controls a rate at which the water that enters the second path passes through the compartment such that the water combines with the solid, water-soluble substance held within the compartment to form the concentrated solution. The device also comprises a nozzle near a second end of the body, wherein the end portion of the interior tubing extends to the nozzle such that the water travelling through the first path of the interior tubing and the concentrated solution travelling through the second path of the interior tubing combines in the end portion of the interior tubing to form the final solution, and wherein the final solution exits the device through the nozzle.

The details of one or more examples of the disclosure are set forth in the accompanying drawings and the description below. Other features, objects, and advantages of the disclosure will be apparent from the description and drawings, and from the claims.

### BRIEF DESCRIPTION OF DRAWINGS

The following drawings are illustrative of particular examples of the present invention and therefore do not limit the scope of the invention. The drawings are not necessarily to scale, though embodiments can include the scale illustrated, and are intended for use in conjunction with the explanations in the following detailed description wherein like reference characters denote like elements. Examples of the present invention will hereinafter be described in conjunction with the appended drawings.

FIG. 1 is a diagram illustrating an example device in accordance with the devices and techniques described herein.

FIG. 2 is an expanded view of an example device in accordance with the devices and techniques described herein.

### DETAILED DESCRIPTION

The following detailed description is exemplary in nature and is not intended to limit the scope, applicability, or configuration of the invention in any way. Rather, the following description provides some practical illustrations for implementing examples of the present invention. Those skilled in the art will recognize that many of the noted examples have a variety of suitable alternatives.

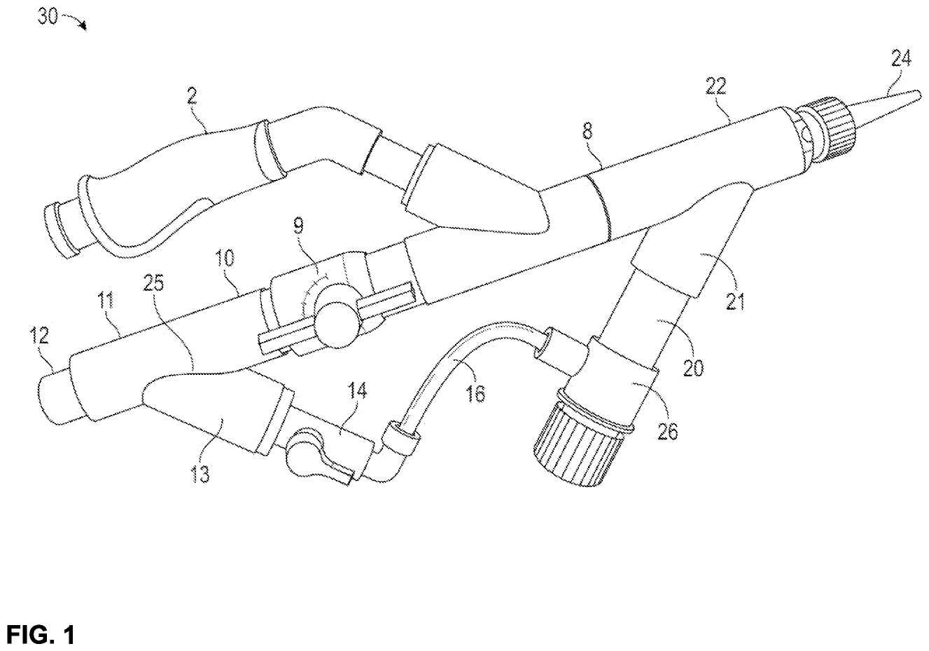

FIG. 1 is a diagram illustrating an example device 30 in accordance with the devices and techniques described herein. In general, device 30 is a handheld device that is configured to attach to a water source, direct water through two separate portions of device 30, one of which holds a solid, water-soluble substance, and then provide a connection to re-combine the water from the two separate portions to create a final solution before the final solution is sprayed out of an end nozzle. Device 30 includes dials on each of the portions to control the amount of water and rate at which water passes over the water-soluble substance, thereby enabling the user to control the concentration of the water-soluble substance in the final solution before it is sprayed out into the user's immediate environment.

As shown in FIG. 1, handle 2 connects to body 8 such that a user can easily control the direction at which liquid is sprayed out of device 30. Device 30 connects to a water source via coupling 12. Interior tubing 16 is spread throughout device 30, either as an explicitly formed tube inserted into parts of device 30 (such as the exposed piece of tubing showed and labelled interior tubing 16) or as merely a hollowed out portion of device 30 that facilitates the transport of water throughout device 30.

After water or any other liquid enters device 30 via coupling 12, the liquid enters base portion 11 of interior tubing 16 of device 30. The liquid initially flows through fork 25 and into first path 10. Upon reaching the portion of body 8 that includes first dial 9, backpressure is created based on how much first dial 9 is rotated to engage an actuator within the portion of body 8 that includes first dial 9. The actuator (or rotating contraption or any component that can block the progression of water through first path 10), as it becomes more engaged to block more liquid in first path 10, creates backpressure to send the liquid back through second path 13 of fork 25.

Second dial 14 of device 30 works similarly to first dial 9, in that the rotation of second dial 14 controls an actuator, rotating contraption, or any other component that can block the progression of water through second path 13 of interior tubing 16 such that second dial 14 controls the amount of liquid and the rate at which liquid passes through second path 13.

Second path 13 includes compartment 20. Compartment 20 is configured to hold solid, water-soluble (SWS) substance 26. As liquid travels along second path 13 and into compartment 20, the liquid passes over SWS substance 26 to create a concentrated solution that is part-liquid and part-SWS substance 26.

The liquid from first path 10 and the concentrated solution in compartment 20 re-combine in end portion 22 of interior tubing 16 to form a final solution that is a more diluted form of the concentrated solution from compartment 20, the final solution still being part-liquid and part-SWS substance 26. Sieve plate 21 may be included to further control, in combination with second dial 14, the rate at which the concentrated solution exits compartment 20 and re-combines with first path 10 in end portion 22.

The combination of first dial 9 and second dial 14 (and, in some instances, sieve plate 21) controls the concentration of concentration of SWS substance 26 in the final solution that exits device 30 via nozzle 24. In this way, device 30 provides a simple solution to users who need to configure the stream exiting nozzle 24 to have a particular concentration in the final solution. For instance, in cleaning and disinfecting applications, where SWS substance 26 is a solid chlorine puck or a solid soap puck, the user may rotate first dial 9 and second dial 14 to get a very specific concentration of chlorine bleach or soap in the stream exiting nozzle 24, enabling the user to use device 30 in any number of environments or for any number of sanitation uses. Furthermore, by configuring device 30 in this way with SWS substance 26 as opposed to a liquid substance, device 30 provides a safer device for cleaning and disinfecting purposes, as solid substances tend to be safer to handle than liquid substances of the same compound.

Device 30 provides a fast, easy and safe device for sanitation of hard surfaces, where the user can dial-in the chlorine level that they desire for cleaning, sanitizing or disinfecting. Device 30 may be powered by normal water pressure (usually between 30 and 80 psi) with various nozzles for gentle sprays to jet streams up to 40 feet. Device 30 may use SWS substances, such as solid chlorine tablets, which provide erosion at predictable rates with highly accurate chlorine levels, eliminating premature dissolving and the safety issues associated with liquid chlorine.

With the ability to alter the concentration of the final solution, device 30 may be used to clean (typically ˜0 to 50 ppm of chlorine), sanitize (typically ˜50 to 200 ppm of chlorine), and disinfect (typically ˜500 to 800 ppm of chlorine) all with the mere rotation of first dial 9 and/or second dial 14. Chlorine test strips may be built into handle 2 or body 8 for easy access and calibration of the concentrations. To operate, SWS substance 26 may be placed in compartment 20, dials 9 and 14 are rotated, and the final solution exits nozzle 24 to spray down the area. If disinfecting at levels above 200 ppm, device 30 could be configured such that it includes only fresh water for a final spray down. In certain conditions, SWS substance 26 may erode in 25-30 minutes, providing adequate time to sanitize your hard surface and eliminating the need to remove and store chlorine tablets. As with all chlorine products, the user may wish to wear protective gear, including gloves, face mask and protective clothing.

SWS substance 26 may be substances for other uses rather than simply cleaning substances. For instance, SWS substance 26 could be a fertilizer tablet for tree and shrub fertilization, or herbicide or pesticide tablets for weed and pest control. Virtually any solid chemical designed for dilution and application can be used and allows the operator to highly control the level of concentrate.

Rather than mixing portions of an already created liquid solution into a single water stream, device 30 allows the user to “dial-in” and control solution levels, such as chlorine levels that they desire. Device 30 also may have built in test strips, including chlorine test strips, so that the user can be assured of proper chemical levels in the solution. Device 30 may use chlorine tablets (e.g., 1″ chlorine tablets, or any other sized tablets) in a closed and metered chamber that assures consistency of chlorine delivery. The solid chlorine tablets deliver chlorine on demand in a closed chamber, thus eliminating fuming and the issues associated with liquid chlorine. For chlorine, the tablets may erode in 25-30 minutes, thus eliminating the need to remove tablets for safe storage after use.

Adding to the complexity of the issue at hand, different water pressures, water temperatures, and different initial water chemistries may influence the rate at which SWS substance 26 may dissolve. Using back pressure (generated by first dial 9), flow control (controlled by second dial 14 and/or sieve plate 21) and a closed and metered chamber (e.g., compartment 20), the user may utilize device 30 to dial-in or calibrate their chemical levels in the solution, such as chlorine, to generate the correct concentration given the user's particular water pressure, water temperature, and water chemistry. Body 8 or handle 2 may also hold chlorine test strips and a color chart for immediate testing of chlorine levels.

Liquid chlorine, purchased by consumers from their local store, has chlorine levels of 5.25% to 8.25%, which is equivalent to 52,500 ppm to 82,500 ppm. Users are asked to dilute the concentrate at ⅓ cup chlorine to 1 gallon of water, thus delivering 1,100 ppm at the 5.25% concentration level. Device 30 eliminates the risks associated with liquid chlorine. Additionally, liquid chlorine begins to lose its potency in a matter of days and weeks, while solid chlorine tablets retain their chlorine levels for months and years.

Chlorine effectiveness is a function of chlorine level and dwell time, or the amount of time the chlorine sits on the hard surface. All levels of cleaning, sanitation, and disinfection can be achieved for levels from 20 ppm to 1,000 ppm. The Center for Disease Control (CDC) states that outdoor hard surfaces only need to be cleaned (using 0-50 ppm chlorine solutions) if they sit unused for 7 days, but that hard surfaces that are frequently touch should be sanitized (50 ppm to 200 ppm chlorine solutions) or disinfected (200 ppm to 1,000 ppm chlorine solutions). These areas include municipalities, pool areas especially with handrails, chairs, and tables, playgrounds, event centers, universities, stadiums, locker rooms, outdoor student areas, hotels and motels, patio areas, restaurants with outdoor seating, amusement parks, animal care facilities (e.g., kennels), meat processing facilities, and vegetable processing.

In accordance with the devices and techniques described herein, device 30 includes body 8. Depending upon the application use, body 8 may be made of any one or more of a number of materials, including polyvinyl chloride (PVC), plastic, rubber, metal, a metal alloy, or a combination thereof. The PVC may be schedule 80 PVC, which is particularly suited for withstanding chlorine at any level of concentration; however, a wide variety of materials could be used for device 30 and body 8. The material may be dependent on the type of substance that is SWS substance 26, and the material may be any material best suited to withstand the chemicals that make up SWS substance 26.

Device 30 may also include coupling 12, which may be configured to attach device 30 to a water source or other liquid source. For instance, coupling 12 may be configured to receive a coupling end of a hose. Coupling 12 may be situated near a first end of body 8 of device 30.

Device 30 may also include interior tubing 16 located inside body 8 either as a specific insert or as a hollowed-portion of body 8. Interior tubing 16 may be any structure or hole that enables the transportation of a liquid throughout body 8. Interior tubing 16 may include base portion 11, fork 25 that separates base portion 11 into first path 10 and second path 13, and end portion 22 that combines first path 10 and second path 13. Base portion 11 of interior tubing 16 receives water into device 30 via coupling 12.

Device 30 may further include compartment 20 along second path 13 that holds a solid, water-soluble (SWS) substance 26. SWS substance 26 may be any one or more of a solid chlorine tablet, a solid soap tablet, a solid fertilizer tablet, a solid herbicide tablet, and a solid pesticide tablet. These different water-soluble materials enable device 30 to have a range of uses. A solid form of any possible SWS substance 26 is safer than liquid, does not require mixing, and can be better controlled by device 30.

Device 30 may also include first dial 9 that, when rotated, controls an amount of the water that enters first path 10 from base portion 11 and an amount of the water that enters second path 13. First dial 9 may control a first actuator in first path 10, where rotating first dial 9 to reduce the amount of water that enters 10 first path extends the actuator into first path 10. The first actuator being extended into first path 10 may create backpressure within first path 10 to direct water into second path 13.

Device 30 may also include second dial 14 that, when rotated, controls a rate at which the water that enters second path 13 passes through compartment 20 such that the water combines with SWS substance 26 held within compartment 20 to form a concentrated solution of water and SWS substance 26. Second dial 14 may control a second actuator in second path 13, where rotating second dial 14 to reduce the rate at which the water that enters second path 13 passes through compartment 20 extends the second actuator into second path 13. A concentration of the concentrated solution may be based at least in part on a rotated position of first dial 9 and a rotated position of second dial 14. Further, a concentration of the final solution may be based at least in part on a rotated position of first dial 9 and a rotated position of second dial 14. A rotated position of second dial 14 may also define a pressure at which the final solution is expelled from nozzle 24.

Device 30 may also include nozzle 24 near a second end of body 8. End portion 22 of interior tubing 16 extends to nozzle 24 such that the water travelling through first path 10 of interior tubing 16 and the concentrated solution travelling through second path 13 of interior tubing 16 combines in end portion 22 of interior tubing 16 to form a final solution. The final solution exits device 30 through nozzle 24. Nozzle 24 may be configured to expel the final solution in any of a plurality of patterns. For instance nozzle 24 may be rotatable and similar to certain multi-pattern hose nozzles, where a rotated position of nozzle 24 may define a pattern of the plurality of patterns to be used in expelling the final solution.

Device 30 may also include other components. For instance, device 30 may include handle 2. Handle 2 may extend from a central portion of body 8 between the first end that includes coupling 12 and the second end that includes nozzle 24.

Device 30 may also include a test strip compartment that stores one or more test strips that measure an actual concentration of SWS substance 26 in the final solution. The test strips may be configured to test for whichever of the substances make up SWS substance 26. For instance, the one or more test strips could include one or more of chorine test strips, soap test strips, fertilizer test strips, herbicide test strips, and pesticide test strips.

Device 30 may also include sieve plate 21 located in compartment 20 between compartment 20 and end portion 22. Sieve plate 21 may include a plurality of holes, with each hole of the plurality of holes in sieve plate 21 having a unique size, shape, or placement. In such instances, sieve plate 21 may be rotatable within compartment 20 such that one or more holes of the plurality of holes is an active hole at any rotated position of the sieve plate. The concentration of the concentrated and solutions, and a flow rate of the final solution at a given time are further based on the one or more active holes at the given time. In other instances, sieve plate 21 may be removable from a sieve plate slot such that a second sieve plate can be inserted into the sieve plate slot to achieve different concentrations and flow rates. Sieve plate 21 may be hard plumbed into device 30, but may also be a twist dial that can change the sieve plate configuration (e.g., number of holes, size of holes, shape of holes and placement). Sieve plate 21 may further meter the flow of water and control the amount of time and circulation in compartment 20 with SWS substance 26. Sieve plate 21 may deliver from 0 ppm to upwards of 800 ppm. With a different sieve plate or an adjustable sieve plate, device 30 can change the concentration levels delivered by device 30 further.

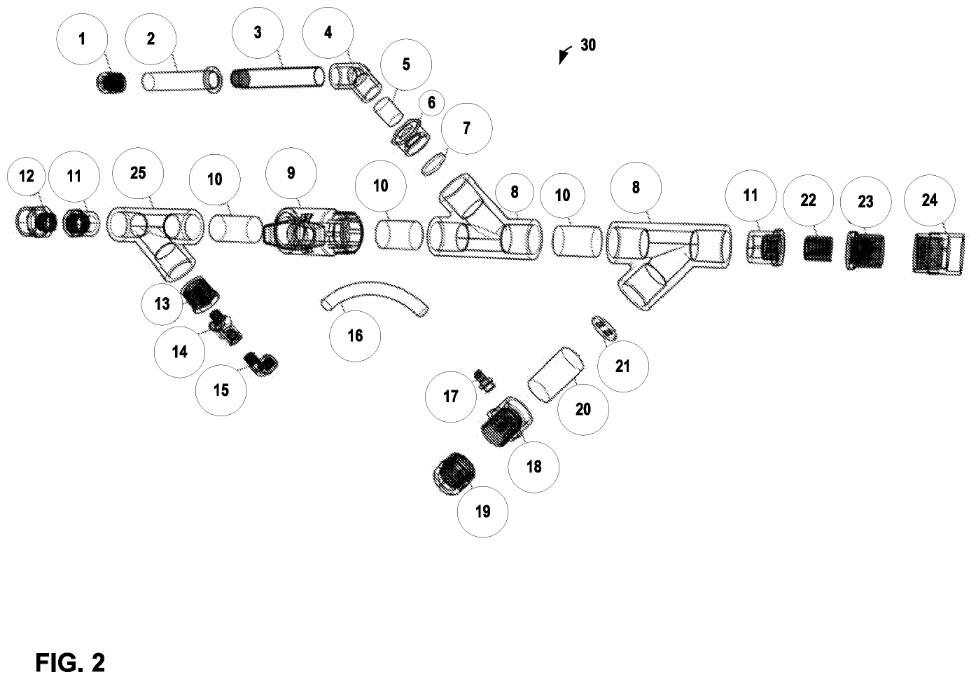

FIG. 2 is an expanded view of an example device in accordance with the devices and techniques described herein. FIG. 2 illustrates only one particular example of device 30, and many other examples of device 30 may be used in other instances and may include a subset of the components included in example device 30 or may include additional components not shown in FIG. 2.

Parts in FIG. 2 with similar numbers as parts in FIG. 1 may have similar compositions, functions, and uses as the parts described above with respect to FIG. 1. For instance, coupling 12 of FIG. 2 may have similar functions as coupling 12 of FIG. 1 to attach device 30 to a water source or other liquid source. Furthermore, to the extent that particular materials, components, or measurements are described in the below parts list, one having ordinary skill in the art would understand that any suitable replacement is contemplated herein. For instance, a different degree bend for elbow 4, first dial 9 may be used with a different valve other than a ball valve, or a different material for interior tubing 16 would be understood as being contemplated herein. Any device that performs the functions as described in the claims could be considered as a viable device under this specification.

The parts shown in FIG. 2 include the following:
1—Cap
2—Handle (e.g., an ergonomic comfort grip)
3—Nipple toe
4—45-degree Elbow
5—Nipple plain
6—Reducer bushing SPG×SOC
7—PVC disk
8—PVC body (shown with a fork to attach handle 2 to body 8)
9—First dial (shown with a 1″ ball valve)
10—First path of PVC piping
11—Base portion of PVC piping shown as a reducer bushing SPG×FPT
12—Coupling shown as a male adapter
13—Second path shown as a reducer bushing SPG×FPT
14—Second dial (shown with a ¼″ ball valve)
15—Elbow tube
16—Clear PVC tubing
17—Tube and Male National Pipe Taper Fuel (NPTF)
18—Male adapter
19—Cap
20—Compartment shown as a clear PVC tube
21—Sieve plate shown with four holes
22—End portion shown is a PVC nipple
23—Reducer bushing MPT×FPT
24—Nozzle (e.g., spray gun nozzle)
25—Fork shown as a PVC element of overall body 8

It is to be recognized that depending on the example, certain acts or events of any of the techniques described herein can be performed in a different sequence, may be added, merged, or left out altogether (e.g., not all described acts or events are necessary for the practice of the techniques). Moreover, in certain examples, acts or events may be performed concurrently, e.g., through multi-threaded processing, interrupt processing, or multiple processors, rather than sequentially.

Various examples of the disclosure have been described. Any combination of the described systems, operations, or functions is contemplated. These and other examples are within the scope of the following claims.

What is claimed is:

1. A device comprising:
a body;
a coupling configured to attach the device to a water source, the coupling near a first end of the body;
interior tubing located inside the body, the interior tubing including a base portion, a fork that separates the base portion into a first path and a second path, and an end portion that combines the first path and the second path, wherein the base portion of the interior tubing receives water into the device via the coupling;
a compartment along the second path that holds a solid, water-soluble substance;
a first dial that, when rotated, controls an amount of the water that enters the first path from the base portion and an amount of the water that enters the second path;
a second dial that, when rotated, controls a rate at which the water that enters the second path passes through the compartment such that the water combines with the solid, water-soluble substance held within the compartment to form a concentrated solution; and
a nozzle near a second end of the body, wherein the end portion of the interior tubing extends to the nozzle such that the water travelling through the first path of the interior tubing and the concentrated solution travelling through the second path of the interior tubing combines in the end portion of the interior tubing to form a final solution, and wherein the final solution exits the device through the nozzle.

2. The device of claim 1, wherein the first dial controls a first actuator in the first path, wherein rotating the first dial to reduce the amount of water that enters the first path extends the actuator into the first path.

3. The device of claim 2, wherein the first actuator being extended into the first path creates backpressure within the first path to direct water into the second path.

4. The device of claim 1, wherein the second dial controls a second actuator in the second path, wherein rotating the second dial to reduce the rate at which the water that enters the second path passes through the compartment extends the second actuator into the second path.

5. The device of claim 1, further comprising a handle.

6. The device of claim 5, wherein the handle extends from a central portion of the body between the first end that includes the coupling and the second end that includes the nozzle.

7. The device of claim 1, wherein the solid, water-soluble substance comprises one or more of a solid chlorine tablet, a solid soap tablet, a solid fertilizer tablet, a solid herbicide tablet, and a solid pesticide tablet.

8. The device of claim 1, wherein a concentration of the concentrated solution is based at least in part on a rotated position of the first dial and a rotated position of the second dial.

9. The device of claim 1, wherein a concentration of the final solution is based at least in part on a rotated position of the first dial and a rotated position of the second dial.

10. The device of claim 1, wherein the body further comprises a test strip compartment that stores one or more test strips that measure an actual concentration of the final solution.

11. The device of claim 10, wherein the one or more test strips comprise one or more of chorine test strips, soap test strips, fertilizer test strips, herbicide test strips, and pesticide test strips.

12. The device of claim 1, wherein the coupling is configured to receive a coupling end of a hose.

13. The device of claim 1, wherein the nozzle is configured to expel the final solution in any of a plurality of patterns, wherein a rotated position of the nozzle defines a pattern of the plurality of patterns to be used in expelling the final solution.

14. The device of claim 1, wherein the body is made of one or more of polyvinyl chloride (PVC), plastic, rubber, metal, a metal alloy, or a combination thereof.

15. The device of claim 1, wherein a rotated position of the second dial further defines a pressure at which the final solution is expelled from the nozzle.

16. The device of claim 1, further comprising a sieve plate located in the compartment.

17. The device of claim 16, wherein the sieve plate comprises a plurality of holes, wherein each hole of the plurality of holes in the sieve plate has a unique size, shape, or placement, wherein the sieve plate is rotatable within the compartment such that one or more holes of the plurality of holes is an active hole at any rotated position of the sieve plate, and wherein the concentration of the concentrated solution, the concentration of the final solution, and a flow rate of the final solution at a given time are further based on the one or more active holes at the given time.

18. The device of claim 16, wherein the sieve plate is removable from a sieve plate slot such that a second sieve plate can be inserted into the sieve plate slot.

19. A system comprising:
a hose;
a solid, water-soluble substance; and
a device comprising:
a body;
a coupling configured to attach the device to the hose, the coupling near a first end of the body;
interior tubing located inside the body, the interior tubing including a base portion, a fork that separates the base portion into a first path and a second path, and an end portion that combines the first path and the second path, wherein the base portion of the interior tubing receives water into the device via the coupling and the hose;
a compartment along the second path that holds the solid, water-soluble substance;
a first dial that, when rotated, controls an amount of the water that enters the first path from the base portion and an amount of the water that enters the second path;
a second dial that, when rotated, controls a rate at which the water that enters the second path passes through the compartment such that the water combines with the solid, water-soluble substance held within the compartment to form a concentrated solution; and
a nozzle near a second end of the body, wherein the end portion of the interior tubing extends to the nozzle such that the water travelling through the first path of the interior tubing and the concentrated solution travelling through the second path of the interior tubing combines in the end portion of the interior tubing to form a final solution, and wherein the final solution exits the device through the nozzle.

20. A method comprising:
rotating one or more of a first dial and a second dial of a device to adjust a concentration of one or more of a concentrated solution and a final solution, wherein the device comprises:
a body;
a coupling configured to attach the device to a water source, the coupling near a first end of the body;
interior tubing located inside the body, the interior tubing including a base portion, a fork that separates the base portion into a first path and a second path, and an end portion that combines the first path and the second path, wherein the base portion of the interior tubing receives water into the device via the coupling;
a compartment along the second path that holds a solid, water-soluble substance;
the first dial, wherein rotating the first dial controls an amount of the water that enters the first path from the base portion and an amount of the water that enters the second path;
the second dial, wherein rotating the second dial controls a rate at which the water that enters the second path passes through the compartment such that the water combines with the solid, water-soluble substance held within the compartment to form the concentrated solution; and
a nozzle near a second end of the body, wherein the end portion of the interior tubing extends to the nozzle such that the water travelling through the first path of the interior tubing and the concentrated solution travelling through the second path of the interior tubing combines in the end portion of the interior tubing to form the final solution, and wherein the final solution exits the device through the nozzle.
